(19) United States
(12) Patent Application Publication
(10) Pub. No.: US 2025/0380860 A1
(43) Pub. Date: Dec. 18, 2025

(54) MEDICAL SYSTEMS, DEVICES, AND RELATED METHODS FOR APPLYING ENERGY, DELIVERING FLUID, AND/OR MANIPULATING TISSUE

(71) Applicant: Boston Scientific Medical Device Limited, Galway (IE)

(72) Inventors: Gagan JAIN, Titilagarh (IN); Shobhit BANSAL, Gurugram (IN); Chanchala, Gurgaon (IN); Monika GARG, Gurugram (IN)

(73) Assignee: Boston Scientific Medical Device Limited, Galway (IE)

(21) Appl. No.: 19/236,251

(22) Filed: Jun. 12, 2025

Related U.S. Application Data
(60) Provisional application No. 63/661,174, filed on Jun. 18, 2024.

(51) Int. Cl. A61B 1/018; A61B 1/00; A61B 1/05; A61B 1/06; A61B 18/00; A61B 18/14

(52) U.S. Cl. CPC A61B 1/018; A61B 1/00137; A61B 18/1492; A61B 1/05; A61B 1/0676; A61B 2018/00083; A61B 2018/00982; A61B 2218/002

(57) ABSTRACT

A medical system includes an insertion device, a medical device, and an end cap. The insertion device includes an insertion shaft with a working channel. The medical device includes a device shaft and a tool. The end cap includes a proximal extension and a cap tip extending distally of the proximal extension. The proximal extension forms an end cap lumen. The device shaft and the tool of the medical device are movably positioned within the working channel of the insertion shaft. The end cap is positioned within a distal portion of the working channel such that the tool and a distal portion of the device shaft are movable through the end cap lumen.

### CROSS-REFERENCE TO RELATED APPLICATIONS

This application claims the benefit of priority to U.S. Provisional Application No. 63/661,174, filed on Jun. 18, 2024, which is incorporated by reference herein in its entirety.

### TECHNICAL FIELD

Various aspects of this disclosure generally relate to medical systems, devices, and related methods for applying energy, delivering fluid, and/or manipulating tissue. Embodiments of the disclosure relate to medical systems, devices, and related methods of treating tissue by (1) applying electrical energy to or into tissue with a distal element of a first medical device, (2) delivering or injecting fluid into, under, and/or around tissue with the distal element of the first medical device, and/or (3) manipulating tissue with a distal element of a second medical device.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, Peroral Endoscopic Myotomy (POEM), etc. In particular, such procedures may be carried out by inserting an insertion device into a subject's body through a surgical incision, or via a natural anatomical orifice (e.g., mouth, vagina, or rectum), and performing the procedure or operation at a treatment site with an auxiliary device inserted through the insertion device. Alternatively, the auxiliary device may be delivered to the treatment site without the insertion device.

In some procedures, a distal portion of a medical device is positioned between layers of tissue to cut, puncture, or perforate one or more layers of tissue or otherwise treat the tissue or the treatment site. In some aspects, a distal portion of the medical device is positioned between the mucosa and the muscularis layers in a portion of the gastrointestinal (GI) tract to reach a target or treatment site. Oftentimes, the positioning is achieved by (1) injecting a lifting agent or fluid into the submucosa to separate the mucosa layer from the muscularis layer and (2) utilizing a cutting knife to cut through the submucosa. In some instances, tissue that has been cut, punctured, or perforated at the treatment site may interfere with additional treatments. These concerns may increase the duration, costs, and risks of the medical procedure.

The systems, devices, and methods of this disclosure may rectify some of the deficiencies described above or address other aspects of the art.

### SUMMARY

Examples of this disclosure relate to, among other things, medical systems, devices, and methods for performing one or more medical procedures. For example, this disclosure relates to medical devices and methods for performing one of more procedures between layers of tissue. Additionally, in some examples, this disclosure relates to medical systems, devices, and methods for delivering energy (e.g., for cutting, cauterizing, perforating, puncturing, or otherwise treating tissue) and/or delivering fluid to a treatment site. Furthermore, in some examples, this disclosure relates to medical systems, devices, and methods for lifting or otherwise manipulating tissue or one or more portions of the treatment site. Each of the examples disclosed herein may include one or more of the features described in connection with any of the other disclosed examples.

In one example, a medical system may include an insertion device, a medical device, and an end cap. The insertion device may include an insertion shaft with a working channel. The medical device may include a device shaft and a tool. The end cap may include a proximal extension and a cap tip extending distally of the proximal extension. The proximal extension may form an end cap lumen. The device shaft and the tool of the medical device may be movably positioned within the working channel of the insertion shaft. The end cap may be positioned within a distal portion of the working channel such that the tool and a distal portion of the device shaft are movable through the end cap lumen.

The medical system may include one or more of the following aspects. A distal end of the cap tip may include a pointed tip. The end cap may include a widened portion positioned longitudinally between the pointed tip and the proximal extension. The widened portion may include a flat distal surface and a tapered proximal surface. The widened portion further may include a flat longitudinal surface between the flat distal surface and the tapered proximal surface. The end cap may include a stop surface at a distal end of the proximal extension. A proximal end of the proximal extension may include one or more tapered sections.

The tool may be an electrode. The electrode may be longitudinally extendable and retractable relative to the device shaft. The electrode may include an electrode lumen configured to be fluidly connected to a fluid source to deliver fluid through the electrode lumen. The electrode may include a shaft portion and a widened distal portion. The widened distal portion may include a larger diameter than the shaft portion. The electrode may be configured to be energized to deliver energy to tissue.

The end cap may be removably coupleable to the distal portion of the working channel via a snap fit. The working channel of the insertion shaft may include a diameter of at least 4.2 mm, and the end cap lumen may include a diameter of at least 2.2 mm. The end cap may be formed of and/or coated with an electrically insulative material. The insertion device may include a lighting device and an imaging device at a distal end of the insertion shaft.

In another aspect, an end cap for a medical device may include a coupling portion and a cap tip. The coupling portion may include a proximal extension and a stop surface at a distal end of the proximal extension. The cap tip may extend distally from the stop surface. The cap tip may include a first portion, a second portion that extends at an angle from the first portion, and a widened portion. The widened portion may be positioned on a portion of the second portion of the cap tip.

The end cap may include one or more of the following aspects. The distal end of the cap tip may include a pointed tip. The end cap may be formed of and/or coated with an electrically insulative material.

In yet another aspect, a medical system may include a medical device and an end cap. The medical device may include a device shaft and an electrode. The electrode may be longitudinal extendable and retractable relative to a distal end of the device shaft. The end cap may be configured to be coupled to a distal end of a lumen of an insertion device. The end cap may include a coupling portion with a proximal extension and a stop surface at a distal end of the proximal extension. The proximal extension may be configured to be positioned within the distal end of the lumen of the insertion device. The end cap may include a cap tip extending distally of the coupling portion. The cap tip of the end cap may include a widened portion positioned longitudinally between a distal end of the cap tip and the stop surface of the coupling portion.

The medical system may include one or more of the following features. The electrode may include an electrode lumen configured to be fluidly connected to a fluid source to deliver fluid through the electrode lumen. The electrode may include a shaft portion and a widened distal portion. The widened distal portion may include a larger diameter than the shaft portion. The electrode may be configured to be energized to deliver energy to tissue. The distal end of the cap tip may include a pointed tip.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1A illustrates a perspective view of an exemplary medical device system that includes a first medical device, a second medical device, and an end cap, and FIG. 1B illustrates an enlarged view of a distal portion of the medical device system, according to aspects of this disclosure.

FIGS. 2A and 2B illustrate respective perspective views of an exemplary end cap of the medical device system of FIGS. 1A and 1B.

FIG. 3 illustrates a partial cross-sectional view of a distal portion of the medical device system of FIGS. 1A and 1B, including the end cap.

FIG. 4 illustrates a longitudinal cross-sectional view of another exemplary end cap.

### DETAILED DESCRIPTION

Examples of the disclosure include systems, devices, and methods for one or more of: facilitating and improving the efficacy, efficiency, and safety of treating and/or manipulating tissue when, for example, applying electrical energy to tissue with an electrode; delivering fluid into, under, and/or around tissue during a medical procedure through the distal end of the electrode; lifting, moving, or otherwise manipulating tissue; and cutting, resecting, or otherwise treating tissue. Aspects of the disclosure may provide the user with the ability to apply electrical energy or heat to tissue using a medical device having an electrode (e.g., as a distal tool), and to deliver fluid into and/or under tissue with the same medical device. Aspects of the disclosure may provide the user with the ability to apply electrical energy or heat and also deliver fluid without having to switch or swap out tools or end effectors. Aspects of the disclosure may help the user penetrate a layer of tissue (e.g., a submucosal layer) to cause perforation or otherwise cut, cauterize, or otherwise treat tissue.

Aspects of the disclosure may help the user cut, resect, or otherwise remove tissue or other material without having to switch or swap out tools or end effectors. Aspects of the disclosure may help the user to lift, move, or otherwise manipulate (e.g., apply traction to) tissue (e.g., tissue that has already been cut, punctured, or perforated) at or adjacent to a treatment site, which may help the user cut, resect, or otherwise remove additional tissue or other material. Aspects of the disclosure may help the user to lift, move, or otherwise manipulate tissue without having to switch or swap out tools or end effectors. Additionally, aspects of the disclosure may help the user perform one or more of these treatments without requiring a larger insertion device and/or swapping out the medical devices, as a larger insertion device and/or swapping out medical devices may increase the procedure duration, may require multiple users, and/or may otherwise negatively affect the procedure. Moreover, aspects of the disclosure may help the user to lift, move, or otherwise manipulate tissue that has already been cut, punctured, or perforated, or otherwise treated, for example, such that that tissue may not interfere with or otherwise inhibit the cutting or treatment of additional tissue or other portions of the treatment site.

Embodiments of the disclosure may relate to systems, devices, and methods for performing various medical procedures and/or treating portions of the large intestine (colon), small intestine, cecum, esophagus, stomach, any other portion of the gastrointestinal (GI) tract, lungs, and/or any other suitable patient anatomy. For example, various aspects of this disclosure may relate to delivering energy to tissue of an organ or a gland to treat lesions (e.g., tumors), infections, and the like. Various embodiments described herein include single-use or disposable medical devices. Some aspects of the disclosure may be used in performing an endoscopic, arthroscopic, bronchoscopic, ureteroscopic, colonoscopic, laparoscopic, gynoscopic, thoracoscopic, cystoscopic, or other type of procedure. For example, the disclosed aspects may be used with endoscopes, duodenoscopes, arthroscopes, bronchoscopes, ureteroscopes, colonoscopes, laparoscopes, thoracoscopes, gastroscopes, cystoscopes, endoscopic ultrasonography (“EUS”) scopes, aspiration scopes, sheaths, catheters, diagnostic or therapeutic tools or devices, or other types of medical devices. One or more of the elements discussed herein could be metallic, plastic, or include a shape memory metal (such as, e.g., Nitinol), a shape memory polymer, a polymer, or any combination of biocompatible materials.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems, devices, or methods discussed herein may be combined and/or used with one or more other aspects of the medical devices or methods discussed herein.

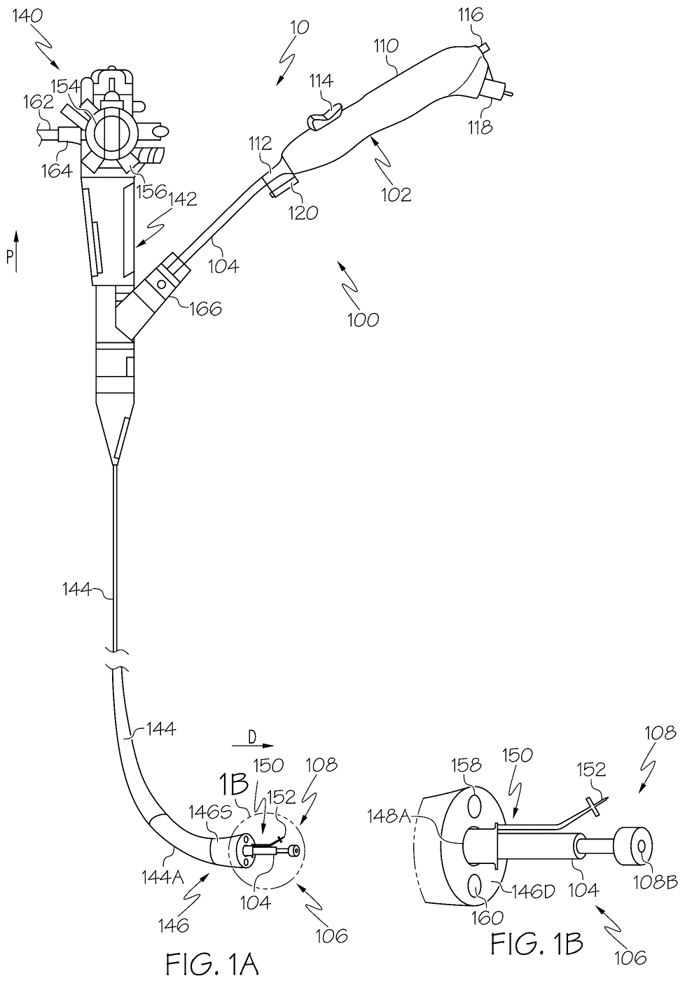

FIG. 1A illustrates a perspective view of an exemplary medical device system 10, including a first exemplary medical device (“medical device 100”) and a second exemplary medical or insertion device (“insertion device 140”), and FIG. 1B illustrates an enlarged view of a distal portion of system 10. Medical device 100 includes a handle 102 and a shaft 104. Shaft 104 includes a distal end 106. Medical device 100 also includes a tool, for example, an electrode 108 at distal end 106. Electrode 108 may be movable (e.g., longitudinally extendable and retractable) relative to shaft 104, for example, extendable and/or retractable longitudinally relative to distal end 106 of shaft 104. Electrode 108 may be energized to cut tissue (e.g., tissue of a bleb or other tissue to be resected at a treatment site). Insertion device 140 includes a handle 142 and a shaft 144.

Additionally, system 10 includes an end cap 150, for example, coupled to or a part of a distal portion of insertion device 140, as discussed in detail below. A proximal portion of end cap 150 may be positioned within a distal end of shaft 144, and a distal portion of end cap 150 may include a cap tip 152 that extends distally of shaft 144. In some aspects, cap tip 152 of end cap 150 may abut, engage, or otherwise interact with tissue that has been cut, punctured, or perforated by electrode 108. As discussed in detail below, cap tip 152 may help to lift or otherwise urge tissue distally and/or radially away from electrode 108, shaft 104, and/or other portions of medical device 100.

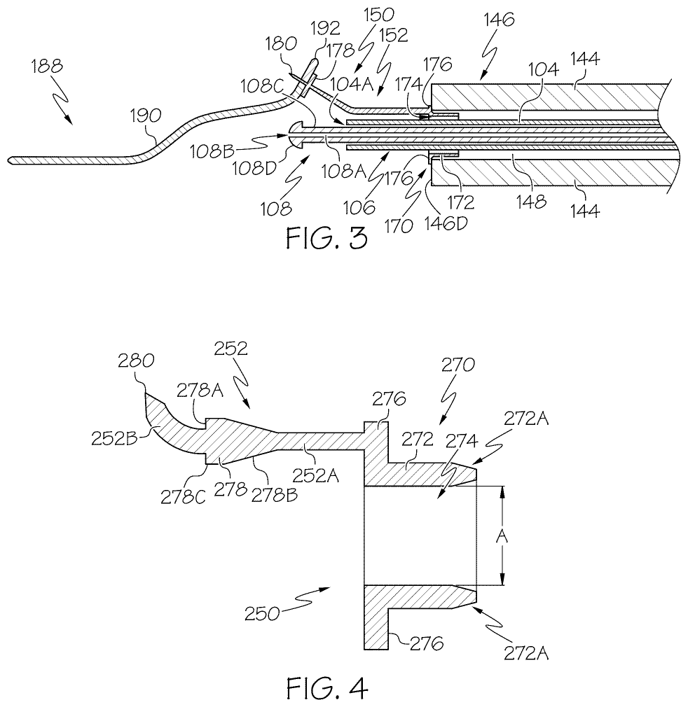

For instance, electrode 108 an electrosurgical knife or other cautery element. Electrode 108 may be energized to puncture, perforate, cut, resect, cauterize, remove, or otherwise treat tissue or other portions of the treatment site. In some aspects, electrode 108 may include a central lumen or an electrode lumen 108A (FIG. 3) that terminates in an electrode outlet or electrode opening 108B to inject or otherwise deliver fluid to the treatment site, for example, into or between layers of tissue (e.g., to help form a bleb). In other aspects, however, electrode 108 may not include an electrode lumen, and thus may not provide for injection or other delivery of fluid. In any of these aspects, a portion of end cap 150 may help to abut, engage, or otherwise interact with tissue that has been cut, punctured, or perforated by electrode 108 and lift or urge the tissue away from electrode 108, shaft 104, other portions of medical device 100, and/or portions of insertion device 140.

As shown in FIG. 1A, handle 102 includes a handle body 110. A distal portion of handle body 110 may be coupled to a proximal portion of shaft 104 via a connector or a strain relief element 112. Additionally, handle 102 includes one or more actuators or control mechanisms. For example, handle 102 may include an actuation mechanism, button, actuator, or trigger 114, for example, movable relative to a distal portion of handle body 110. Trigger 114 may be movable, for example, slidable (e.g., longitudinally movable), actuatable, or depressible, to control a position (e.g., extension and/or retraction) and/or energization of electrode 108. For example, sliding, actuating, or depressing trigger 114 may extend electrode 108 from distal end 106 of shaft 104. In these aspects, electrode 108 may be extendable from distal end 106 of shaft 104 by approximately 5.0 mm to approximately 2.0 cm. Trigger 114 may be biased (e.g., spring-biased), such that trigger 114 may return to the proximal or non-depressed position once pressure on trigger 114 is no longer applied. Returning to the proximal or non-depressed position may also retract electrode 108 to the non-extended position. Alternatively or additionally, in some aspects, sliding, actuating, or depressing trigger 114 may control the energization of electrode 108. For example, depressing trigger 114 may energize electrode 108, and releasing pressure on trigger 114 may terminate the energizing of electrode 108. In other aspects, the energization of electrode 108 may be controlled by a different button, actuator, or trigger, for example, via a foot pedal or other control unit.

Handle 102 also includes one or more connections and/or ports, for example, extending from or otherwise coupled to handle body 110. For example, handle 102 may include one or more of an electrode fluid port 116 and/or an electrical connection 118. As discussed below, electrode fluid port 116 may be fluidly connected to electrode 108, for example, via one or more lumens in handle 102 and shaft 104. Electrical connection 118 may be electrically connected to electrode 108, for example, via one or more conductive elements (e.g., wires, cables, filaments, rods, etc.) in handle 102 and shaft 104.

Electrode fluid port 116 and/or electrical connection 118 may be positioned on respective proximal portions of handle body 110. Electrode fluid port 116 may receive fluid (e.g., water, saline, ORISE™ gel, other lifting agent(s), etc.) to be delivered via a lumen in electrode 108, for example, between two layers of tissue or otherwise to the treatment site. The delivery of the fluid to each of electrode fluid port 116 may be controlled by a fluid delivery device, for example, a syringe, pump, etc. Moreover, electrical connection 118 may receive energy (e.g., cautery energy) from an energy source to be delivered to electrode 108, for example, to cut, cauterize, penetrate, perforate, or otherwise treat tissue. In some aspects, the delivery of energy to electrical connection 118 may be controllable, for example, via a separate button or trigger, one or more foot pedals, etc.

In some aspects, handle 102 may include a cleaning tool 120, for example, coupled to a distal portion of handle 102 and/or strain relief element 112. In some aspects, cleaning tool 120 may include an internal pin or extension (not shown), and cleaning tool may receive a portion of electrode 108 to help clean a distal portion of electrode 108 (e.g., the internal channel or lumen within electrode 108 and/or an external surface of electrode 108).

Furthermore, although not shown, handle 102 may include a strap or other connection element, for example, coupled to or otherwise extending from handle body 110. In some aspects, the strap or other connection element may help to allow for handle body 110 to be coupled to another medical device, for example, to a handle or other portion of a scope or other insertion device (e.g., handle 142 of insertion device 140).

Insertion device 140 may be an endoscope, a duodenoscope, or another insertion device. In these aspects, insertion device 140 includes handle 142 and shaft 144. Shaft 144 includes a distal end 146. Shaft 144 includes at least one lumen 148 (FIG. 3) that terminates at distal end 146 in a distal opening 148A. Shaft 144 may include an articulation portion 144A, for example, proximal of distal end 146.

Handle 142 may include one or more actuating devices or actuators configured to articulate or otherwise move articulation portion 144A in one or more directions, thereby moving distal end 146 in one or more directions. For example, a plurality of actuating elements, such as cables or wires, may extend distally from a proximal end of insertion device 140 (e.g., such as handle 142), through one or more lumens of shaft 144, to articulation portion 144A and/or distal end 146. For example, the actuating elements may be directly or indirectly coupled to first and second actuating devices 154, 156, which may control articulation of articulation joint 16 in multiple directions, such as up, down, left, and/or right. Actuating devices 154, 156 may be, for example, rotatable knobs that rotate about their axes to push/pull actuating elements extending through insertion device 140. In some aspects, handle 142 may include one or more locking mechanisms to help secure the position of one or more of actuating devices 154, 156, and thus help to secure the position of articulation portion 144A and/or distal end 146.

Additional devices (e.g., knobs, buttons, levers, etc.) may be configured to control other aspects of insertion device 140. For example, additional devices may control aspects of distal end 146, and/or aspects of elements attached to distal end 146. In some aspects, the additional devices may control up/down movement of an elevator (not shown) of distal end 146.

Distal end 146 may include one or more imaging devices 158 (e.g., camera) and/or one or more lighting devices 160 (e.g., LEDs, optical fibers, etc.) disposed on a distalmost face 146D of distal end 146. Although not shown, in some aspects, imaging device 158 and/or lighting device 160 may, additionally or alternatively, be disposed on a side surface 146S of distal end 146 (e.g., proximal to distalmost face 146D of distal end 146). Furthermore, although imaging device 158 and lighting device 160 are shown on opposing sides of distal opening 148A, this disclosure is not so limited. For example, imaging device 158 and lighting device 160 may be positioned on a same side (e.g., adjacent to one another) of distal opening 148A. Furthermore, one or more users may utilize imaging device 158 and/or lighting device 160 to visualize one or more of electrode 108, distal end 106 of shaft 104, end cap 150, and/or a treatment site.

Although not shown, one or more electrical cables or wires may extend from the proximal end of insertion device 140 (e.g., handle 142), through shaft 144, and to imaging device(s) 158 and/or lighting device(s) 160. The electrical cables or wires may provide electrical power and/or controls to imaging device 158, lighting device(s) 160, and/or other electrical devices in or on distal end 146. In aspects, the electrical cables or wires may carry imaging signals from distal end 146 proximally, for example, to be processed by a controller and/or displayed on a display.

As mentioned above, distal end 146 may further include one or more openings 148A disposed on distalmost face 146D. Opening(s) 148A may additionally or alternatively be disposed on side surface 146S of distal end 146. Opening(s) 148A may be distal openings to one or more lumens 148 (e.g., a working channel) extending through shaft 144.

In some aspects, system 10 may include an umbilicus 162, for example, to couple a portion (e.g., handle 142) of insertion device 140 to one or more other components. A connector or strain relief 164 may assist in attaching umbilicus 162 to handle 142. Umbilicus 162 may be configured for introducing fluid, suction, and/or wiring for electronic components to handle 142. Umbilicus 162 may include a connector (e.g., for connecting umbilicus 162 to one or more of a controller, a display, etc., not shown).

Handle 142 may further include a port 166 for introducing and/or removing tools, fluids, devices, and/or other materials to and/or from the subject. For example, port 166 may be in fluid communication with one or more lumens 148 (e.g., the working channel) of shaft 144 and distal end 146. In these aspects, port 166 may be configured to receive a portion of medical device 100. For example, a portion (e.g., a portion of shaft 104) of medical device 100 may be inserted through port 166 and lumen 148 of shaft 144. Distal end 106 of shaft 104 of medical device 100 may be extended distally from distal end 146, for example, via distal opening 148A. Shaft 104 of medical device 100 may be movable (e.g., translatable proximally and/or distally) within lumen 148 of insertion device 140, for example, controlling a length of medical device 100 that extends distally of distal opening 148A.

Although endoscopes and duodenoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope or endoscope herein should be understood to encompass any of the above medical devices.

As mentioned above, end cap 150 includes a cap tip 152. A proximal portion of end cap 150 may be positioned within or otherwise received within distal opening 148A of lumen 148 of shaft 144 of insertion device 140. For example, the proximal portion of end cap 150 may be coupled (e.g., removably coupled) to distal opening 148A of lumen 148 via a snap fit, press fit, friction fit, or other coupling mechanism. Additionally, at least a portion (e.g., a distal portion) of cap tip 152 may curve or extend radially outward. For example, when end cap 150 is coupled to shaft 144 and when medical device 100 is inserted through insertion device 140, a portion of cap tip 152 may away from shaft 104 of medical device 100 and/or electrode 108. Furthermore, shaft 104 of medical device 100 and/or electrode 108 may be movable (e.g., longitudinally movable) through both lumen 148 and end cap 150. As discussed in detail below, electrode 108 may be positioned and/or manipulated to help inject fluid (e.g., via electrode fluid port 116, lumens in handle 102 and shaft 104, and out of electrode opening 108B) and/or be energized to help cut, puncture, or perforate tissue. In these aspects, end cap 150, including cap tip 152, may help to lift, apply traction to, or otherwise urge the cut, punctured, or perforated tissue distally and/or radially away from electrode 108, shaft 104, and/or other portions of medical device 100.

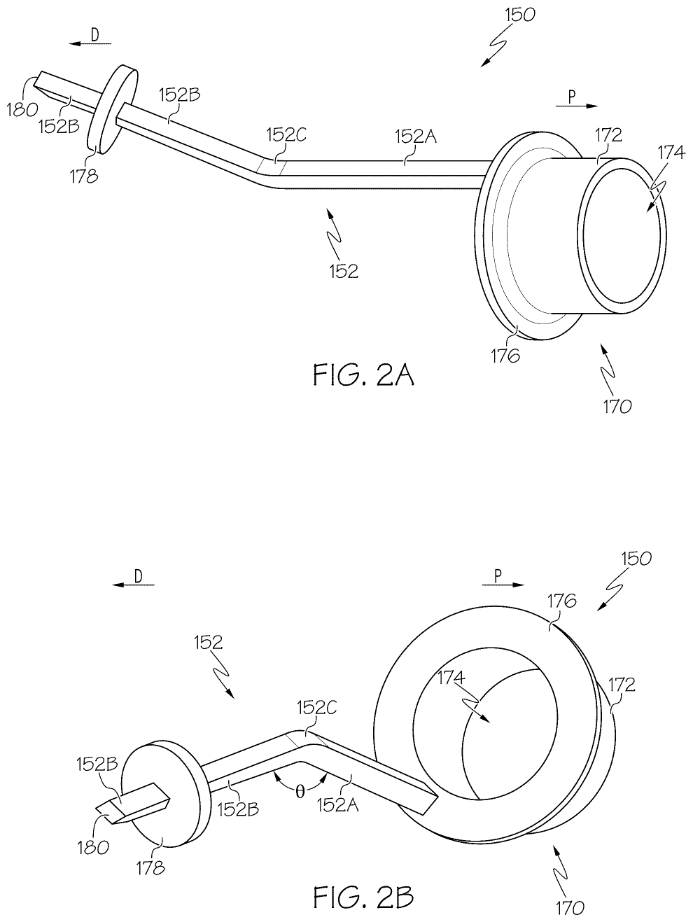

FIGS. 2A and 2B are perspective views of end cap 150. As shown, end cap 150 includes a coupling portion 170 at a proximal end of end cap 150. Coupling portion 170 may include a proximal extension 172. Proximal extension 172 may be generally cylindrical, and may be configured to be received within a lumen of a medical device. For example, as shown in FIG. 3, proximal extension 172 may be received within a distal portion of lumen 148 of shaft 144 of insertion device 140. Proximal extension 172 may form a snap fit, press fit, friction fit, or other coupling with lumen 148, for example, to help couple end cap 150 to shaft 144. Additionally, proximal extension 172 includes or otherwise forms an end cap lumen 174, which may be generally cylindrical. When end cap 150 is coupled to shaft 144, end cap lumen 174 may be generally concentric with (i.e., within) the distal portion of lumen 148. Additionally, end cap lumen 174 may be large enough to movably receive a portion of a medical device. As shown in FIG. 3, end cap lumen 174 may movably receive a distal portion of medical device 100, for example, a distal portion of shaft 104 and electrode 108. In some aspects, end cap lumen 174 may include an inner diameter of at least approximately 2.0 mm, for example, approximately 2.2 mm or greater. In some aspects, lumen 148 of shaft 144 of insertion device 140 may include a diameter of approximately 4.0 mm, for example, approximately 4.2 mm.

In some aspects, one or more portions of end cap 150 may include or form a stop surface 176. For example, a distal portion of coupling portion 170 may include or form stop surface 176. In some aspects, stop surface 176 may be a flat distal face of coupling portion 170, for example, adjacent to a distal end of proximal extension 172. As shown in FIGS. 2A and 2B, stop surface 176 may be wider than proximal extension 172, for example, including a larger outer diameter than proximal extension 172. For example, stop surface 176 may be a flange. Additionally, as shown in FIG. 3, a proximal face of stop surface 176 may at least partially abut distalmost face 146D of shaft 144 insertion device 140 when end cap 150 is coupled to shaft 144. In these aspects, stop surface 176 may help to control the positioning of end cap 150 relative to shaft 144 when coupling end cap 150 to distal end 146.

Moreover, cap tip 152 extends distally from coupling portion 170, for example, substantially perpendicularly from the distal face of stop surface 176. Cap tip 152 may be in the form of a bar or rod. For example, cap tip 152 may include a first portion 152A and a second portion 152B, and second portion 152B may extend distally at a non-zero angle from first portion 152A. In this aspects, cap tip 152 may include a bent portion 152C between first portion 152A and second portion 152B. Second portion 152B may form an angle θ (labeled in FIG. 2B) with first portion 152A. In some aspects, angle θ may be an obtuse angle, for example, approximately 100 degrees to approximately 170 degrees, approximately 115 degrees to approximately 150 degrees, for example, approximately 135 degrees.

Second portion 152B of cap tip 152 may include or be coupled to a widened portion 178. For example, a distal portion of second portion 152B may extend distally of widened portion 178. As shown in FIGS. 2A and 2B, widened portion 178 may be generally cylindrical, washer, or flange shaped. Alternatively, although not shown, widened portion 178 may be one or more other shapes, for example, including triangular, rectangular, pentagonal, or other geometrically shaped surfaces. Furthermore, in other aspects, widened portion 178 may be generally spherical or one or more other shapes (e.g., non-geometric or irregular shapes).

Moreover, a distal end of second portion 152B may include, form, or otherwise be coupled to a pointed, narrowed, or otherwise tapered end tip 180. Although end tip 180 is shown as including a point or sharp end, one or more portions of end cap 150 (e.g., including end tip 180) may be coated in one or more atraumatic coatings, surface finishes, surface features, etc. Additionally or alternatively, one or more portions of end cap 150 (e.g., including end tip 180) may be formed of an atraumatic and/or flexible material, for example, Nitinol, a plastic material, a steel (e.g., stainless steel), or other biocompatible material. In these aspects, end cap 150 may help to lift, apply traction to, move, or otherwise manipulate tissue (e.g., tissue that has already been cut), without inadvertently lifting, moving, or otherwise manipulating unintended tissue.

FIG. 3 illustrates a partial cross-sectional view of distal end 146 of shaft 144 of insertion device 140 (FIGS. 1A and 1B), along with distal end 106 of shaft 104 of medical device 100 (FIGS. 1A and 1B) and end cap 150, relative to a lesion 190 or other tissue at a treatment site 188. As shown, a lesion portion 192 of lesion 190 may have been cut or otherwise separated from the rest of lesion 190 and/or treatment site 188. For example, as mentioned above, electrode 108 may be energized to cut tissue. In some aspects, electrode 108 may deliver one or fluids to help separate tissue layers and/or otherwise manipulate lesion 190 or other portions of treatment site 188.

As shown, end cap 150 may be coupled to distal end 146 of shaft 144. For example, as mentioned above, proximal extension 172 may be positioned within the distal portion of lumen 148. Additionally, stop surface 176 may abut distalmost face 146D of distal end 146. Cap tip 152 may extend distally of stop surface, as discussed above.

Furthermore, end cap 150 includes lumen 174, and a portion of medical device 100 may be movably positioned within lumen 174. For example, shaft 104 and/or electrode 108 may be movably positioned within lumen 174. In these aspects, shaft 104 and/or electrode 108 may be movable (e.g., longitudinally movable proximally and/or distally) through lumen 174. Additionally, when shaft 104 and/or electrode 108 are extended distally of lumen 174, one or more portions of cap tip 152 (e.g., second portion 152B (FIGS. 2A and 2B), widened portion 178, and cap tip 180) may extend radially away from shaft 104 and/or electrode 108, for example, to help lift, apply traction to, move, or otherwise manipulate tissue (e.g., lesion portion 192). For example, as shown in FIG. 3, widened portion 178 and/or cap tip 180 may at least partially abut, engage, or otherwise interact with lesion portion 192. In some aspects, cap tip 180 may extend through a portion of lesion portion 192 to help lift, move, or otherwise manipulate lesion portion 192 or other cut tissue at treatment site 188. Widened portion 178 may help to inhibit cap tip 180 from extending through more tissue than desired, keeping the tissue separated from distalmost face 146D. In these aspects, end cap 150 may help to ensure that shaft 104 and/or electrode 108 do not inadvertent lift, move, or otherwise manipulate unintended tissue, while also allowing shaft 104 and/or electrode 108 to extend distally of shaft 144, for example, such that shaft 104 and/or electrode 108 may cut, cauterize, or otherwise treat the remaining portions of lesion 190, for example, to separate lesion 190 from the remainder of treatment site 188.

As mentioned above, electrode 108 may include electrode lumen 108A, and electrode lumen 108A may be fluidly connected to electrode fluid port 116 (FIG. 1), for example, to deliver one or more fluids through electrode opening 108B and into, under, or between one or more layers of tissue. Additionally, in some aspects, electrode 108 may include an electrode shaft portion 108C and a widened distal portion 108D. Widened distal portion 108D may be wider (e.g., extend radially outward relative to a longitudinal axis) than electrode shaft portion 108C. For example, widened distal portion 108D may be wider a distal opening 104A in the lumen of shaft 104. Widened distal portion 108D may be generally cylindrical (FIGS. 1A and 1B), may be a half-ball or dome shape (FIG. 3), or may be another shape.

FIG. 4 illustrates a longitudinal cross-sectional view of another exemplary end cap 250. As shown, end cap 250 includes a cap tip 252, for example, extending distally from a coupling portion 270. Coupling portion 270 may include a proximal extension 272, which may be configured to be received within a lumen of a medical device, as discussed above. Proximal extension 272 may form an end cap lumen 274, for example, to movably receive one or more medical devices. For example, end cap lumen 274 may include an inner diameter A, for example, at least approximately 2.0 mm, for example, approximately 2.2 mm. Additionally, a distal portion of coupling portion 270 may include or form a stop surface 276, for example, to engage, abut, or otherwise interact with a distal end of a medical device. In some aspects, the proximal end of proximal extension 272 may include one or more tapered sections 272A, for example, with the radially outer wall tapering radially inward and/or the radially inner wall tapering radially outward. Tapered sections 272A may help ease the insertion of the proximal end of end cap 250 (e.g., the proximal end of proximal extension 272) into the distal end of the lumen of the medical device.

Moreover, cap tip 252 extends distally from coupling portion 270, for example, substantially perpendicularly from the distal face of stop surface 276. Cap tip 252 may be in the form of a bar or rod. Cap tip 252 may include a first portion 252A and a second portion 252B, and second portion 252B may extend distally at an angle or curve from first portion 252A. Cap tip 252 may include or be coupled to a widened portion 278, for example, between or in a transition portion between first portion 252A and second portion 252B. Widened portion 278 may include a flat distal surface 278A. Widened portion 278 may also include a slanted or tapered proximal surface 278B. As discussed above with widened portion 178, widened portion 278 and/or flat distal surface 278A may help to inhibit cap tip 280 from extending through more tissue than desired, keeping the tissue separated from the distal portion of an insertion device coupled to end cap 250. Tapered proximal surface 278B may help to form a smooth transition between widened portion 278 and first portion 252A of cap tip 252 and/or help to prevent inadvertent contact with tissue or other aspects of a treatment site. In some aspects, tapered proximal surface 278B may abut or terminate at flat distal surface 278A. In other aspects, as shown, widened portion 278 may include a flat longitudinal surface 278C, for example, between flat distal surface 278A and tapered proximal surface 278B.

Moreover, a distal end of second portion 252B may include, form, or otherwise be coupled to a pointed, narrowed, or otherwise tapered end tip 280. Although end tip 280 is shown as including a point or sharp end, one or more portions of end cap 250 (e.g., including end tip 280) may be coated in one or more atraumatic coatings, surface finishes, surface features, etc. Additionally or alternatively, one or more portions of end cap 250 (e.g., including end tip 280) may be formed of an atraumatic and/or flexible material, for example, Nitinol, a plastic material, a steel (e.g., stainless steel), or other biocompatible material. In these aspects, end cap 250 may help to lift, move, or otherwise manipulate tissue (e.g., tissue that has already been cut, punctured, or perforated), without inadvertently lifting, moving, or otherwise manipulating unintended tissue or other portions of a treatment site.

In any of the aspects discussed here, end caps 150, 250 may be coupled to a medical device, an insertion device, a scope, etc., and may help to form an end effector or a tissue manipulator. For example, end caps 150, 250 may be coupled to a lumen of a medical device, an insertion device, a scope, etc. without any additional coupling mechanisms or features.

Additionally, at least a distal portion of end caps 150, 250 may be biased radially outward (e.g., away from electrode 108 in FIG. 3) to help urge tissue away from medical device 100 (e.g., away from electrode 108). In some aspects, end caps 150, 250 may be formed of one or more materials, for example, Nitinol, a plastic material, a steel (e.g., stainless steel), etc. For example, at least a distal portion of end caps 150, 250 may be able to deform, such that end caps 150, 250 may deform during the delivery and/or positioning of medical system 10 to a treatment site. In these aspects, at least a portion of end caps 150, 250 may be formed of a steel, and may be stamped into the desired pre-set shape. In some other aspects, at least a portion of end caps 150, 250 may be formed of a shape memory material (e.g., Nitinol, plastic, etc.), and may be shape set into the desired pre-set shape.

Furthermore, one or more portions of end caps 150, 250 may be insulative or non-conductive (e.g., formed of a plastic, elastomer, etc.). In some aspects, one or more portions of or an entirety of end caps 150, 250 may be formed of a plastic or other material that is not electrically conductive. In another example, one or more portions of or an entirety of end caps 150, 250 may be coated in an insulative or otherwise non-conductive material (e.g., a dielectric coating). In some examples, end caps 150, 250 may be formed of a metallic material (e.g., stainless steel, Nitinol, etc.), for example, to impart a desired shape, stiffness (e.g., for when pushing on or otherwise interacting with tissue), shape memory, etc., and end caps 150, 250 may be coated with a coating or material that is not electrically conductive (e.g., the dielectric coating). In other examples, end caps 150, 250 may be formed of a plastic or other material that is not electrically conductive. In these aspects, energy delivered to a treatment site (e.g., via electrode 108) may not be conveyed proximally and/or otherwise applied to the treatment site (e.g., other portions or locations of the treatment site or adjacent to the treatment site) via end caps 150, 250 and/or shaft 144 of insertion device 140.

In some aspects, insertion device 140 (e.g., including end caps 150, 250) may be rotatable, for example, along with the rotation of medical device 100 or separately rotatable from medical device 100. In examples, insertion device 140 may be rotatable relative to medical device 100. For example, medical device 100 may be secured to or otherwise coupled to insertion device 140, and medical device 100 and insertion device 140 may be rotated together, thus rotating shaft 104 (including distal end 106), electrode 108, shaft 144, end cap 150, etc. In other aspects, medical device 100 may not be secured from insertion device 140, and medical device 100 may be rotated separately from insertion device 140. In these aspects, shaft 104 (including distal end 106) and electrode 108 may be rotated separately from shaft 144 and end cap 150, and vice versa.

Although not shown, end caps 150, 250 may include one or more other shapes or sizes, which may help end caps 150, 250 push, engage with, or otherwise manipulate tissue at a treatment site. The various shapes and/or configurations of the various end caps 150, 250 may help to promote a grip on tissue when engaged with tissue (e.g., cut tissue of lesion 190). For example, the distal ends (e.g., the distal end faces) of end caps 150, 250 may include one or more points, gripping features (e.g., surface texture, spikes, bumps, etc.), or other features to catch or otherwise interact with and/or manipulate tissue. Additionally or alternatively, the various shapes and/or configurations of end caps 150, 250 may help to promote a shared or abutting surface area when engaged with the tissue, for example, to help push, urge. or otherwise manipulate the tissue away from electrode 108 or other aspects of medical device 100.

Various aspects discussed herein may allow for one or more medical devices (e.g., medical device 100 and insertion device 140) to be delivered to a treatment site, for example, alone or via a scope or insertion device (e.g., insertion device 140), to perform endoscopic submucosal dissection (“ESD”) or otherwise treat a treatment site. Various aspects discussed herein may allow for a user to engage with or otherwise manipulate tissue (e.g., cut tissue) at the treatment site, which may help the user to further cut, puncture, perforate, cauterize, or otherwise treat the treatment site with a reduced risk of otherwise inadvertently contacting unintended tissue or unintended portions of the treatment site. Various aspects discussed herein may help to allow for the user to more effectively or efficiently apply energy, cut, puncture, perforate, cauterize, or otherwise treat tissue, for example, with a reduced likelihood of cut, punctured, or perforated tissue or other aspects of the treatment site interfering with the treatment. Additionally, various aspects discussed herein may help to improve the efficacy of treatment and/or recovery from a procedure, for example, a procedure to treat a treatment side. Various aspects discussed herein may help to reduce and/or minimize the duration of the procedure, and/or may help reduce risks of inadvertent contact with tissue or other material during delivery, repositioning, or removal of a medical device for the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical system, comprising:
an insertion device, wherein the insertion device includes an insertion shaft with a working channel;
a medical device, wherein the medical device includes a device shaft and a tool; and
an end cap, wherein the end cap includes a proximal extension and a cap tip extending distally of the proximal extension, and wherein the proximal extension forms an end cap lumen,
wherein the device shaft and the tool of the medical device are movably positioned within the working channel of the insertion shaft, and
wherein the end cap is positioned within a distal portion of the working channel such that the tool and a distal portion of the device shaft are movable through the end cap lumen.

2. The medical system of claim 1, wherein a distal end of the cap tip includes a pointed tip.

3. The medical system of claim 2, wherein the end cap includes a widened portion positioned longitudinally between the pointed tip and the proximal extension.

4. The medical system of claim 3, wherein the widened portion includes a flat distal surface and a tapered proximal surface.

5. The medical system of claim 4, wherein the widened portion further includes a flat longitudinal surface between the flat distal surface and the tapered proximal surface.

6. The medical system of claim 1, wherein the end cap includes a stop surface at a distal end of the proximal extension.

7. The medical system of claim 6, wherein a proximal end of the proximal extension includes one or more tapered sections.

8. The medical system of claim 1, wherein the tool is an electrode, and wherein the electrode is longitudinally extendable and retractable relative to the device shaft.

9. The medical system of claim 8, wherein the electrode includes an electrode lumen configured to be fluidly connected to a fluid source to deliver fluid through the electrode lumen.

10. The medical system of claim 8, wherein the electrode includes a shaft portion and a widened distal portion, and wherein the widened distal portion includes a larger diameter than the shaft portion.

11. The medical system of claim 8, wherein the electrode is configured to be energized to deliver energy to tissue.

12. The medical system of claim 1, wherein the end cap is removably coupleable to the distal portion of the working channel via a snap fit.

13. The medical system of claim 1, wherein the working channel of the insertion shaft includes a diameter of at least 4.2 mm, and wherein the end cap lumen includes a diameter of at least 2.2 mm.

14. The medical system of claim 1, wherein the end cap is formed of and/or coated with an electrically insulative material.

15. The medical system of claim 1, wherein the insertion device includes a lighting device and an imaging device at a distal end of the insertion shaft.

16. An end cap for a medical device, the end cap comprising:
a coupling portion, wherein the coupling portion includes a proximal extension and a stop surface at a distal end of the proximal extension; and
a cap tip, wherein the cap tip extends distally from the stop surface, wherein the cap tip includes a first portion, a second portion that extends at an angle from the first portion, and a widened portion, wherein the widened portion is positioned on a portion of the second portion of the cap tip.

17. The end cap of claim 16, wherein the distal end of the cap tip includes a pointed tip.

18. The end cap of claim 16, wherein the end cap is formed of and/or coated with an electrically insulative material.

19. A medical system, comprising:
a medical device, wherein the medical device includes a device shaft and an electrode, wherein the electrode is longitudinal extendable and retractable relative to a distal end of the device shaft; and
an end cap configured to be coupled to a distal end of a lumen of an insertion device,
wherein the end cap includes a coupling portion with a proximal extension and a stop surface at a distal end of the proximal extension, wherein the proximal extension is configured to be positioned within the distal end of the lumen of the insertion device, and
wherein the end cap includes a cap tip extending distally of the coupling portion, wherein the cap tip of the end cap includes a widened portion positioned longitudinally between a distal end of the cap tip and the stop surface of the coupling portion.

20. The medical system of claim 19, wherein the electrode includes an electrode lumen configured to be fluidly connected to a fluid source to deliver fluid through the electrode lumen, wherein the electrode includes a shaft portion and a widened distal portion, wherein the widened distal portion includes a larger diameter than the shaft portion, and wherein the electrode is configured to be energized to deliver energy to tissue, and
wherein the distal end of the cap tip includes a pointed tip.
